(19) United States
(12) Patent Application Publication
(10) Pub. No.: US 2019/0029515 A1
(43) Pub. Date: Jan. 31, 2019

(54) Ophthalmological Analysis Method And Analysis System

(71) Applicant: Oculus Optikgeraete GmbH, Wetzlar-Dutenhofen (DE)

(72) Inventors: Gert Koest, Hannover (DE); Andreas Steinmueller, Wettenberg (DE)

(21) Appl. No.: 16/035,224

(22) Filed: Jul. 13, 2018

Related U.S. Application Data
(63) Continuation of application No. 13/463,396, filed on May 3, 2012, now abandoned.

(30) Foreign Application Priority Data
May 31, 2011 (DE) 10 2011 076 793.2

(51) Int. Cl. A61B 3/16; A61B 3/107

(52) U.S. Cl. CPC A61B 3/165; A61B 3/107

(57) ABSTRACT

An ophthalmological analysis method measures an intraocular pressure in an eye using an analysis system. The analysis system includes an actuation device, with which a cornea of the eye is deformed contactlessly. The actuation device applies a puff of air to the eye to deform the cornea. A monitoring system monitors the deformation of the cornea and records sectional images of the undeformed and deformed cornea. An analysis device derives an intraocular pressure from the sectional images of the cornea, wherein the recorded sectional images of the deformed cornea are corrected relative to a recorded sectional image of the undeformed cornea, the intraocular pressure being derived under consideration of the correction.

### CROSS REFERENCE TO RELATED APPLICATION

This application is a continuation of U.S. patent application Ser. No. 13/463,396 filed May 3, 2012, which claims the benefit of German Patent Application No. 10 2011 076 793.2 filed May 31, 2011, each of which is fully incorporated herein by reference.

### STATEMENT REGARDING FEDERALLY SPONSORED RESEARCH OR DEVELOPMENT

Not applicable.

### FIELD OF THE INVENTION

The invention relates to an ophthalmological analysis method for measuring an intraocular pressure in an eye using an analysis system, and to an analysis system of this type formed of an actuation device with which a cornea of the eye is deformed contactlessly, a puff of air being applied to the eye using the actuation device to deform the cornea, formed of a monitoring system with which the deformation of the cornea is monitored and recorded, sectional images of the undeformed and deformed cornea being recorded using the monitoring system, and formed of an analysis device with which the intraocular pressure is derived from the sectional images of the cornea.

### BACKGROUND OF THE INVENTION

Analysis methods and systems of this type are known sufficiently and are used primarily to measure an intraocular pressure in an eye, contactlessly and as accurately as possible. For example, a non-contact tonometer is used for this purpose, with the aid of which a puff of air is applied to the eye to be examined, a strength of the puff of air being selected in such a way that the cornea of the eye is pressed inwards, forming a concave surface shape. Before maximum deformation of the cornea is achieved, and before the cornea folds inwardly towards the ocular lens, the cornea briefly forms a planar surface which is called the “first applanation point”. Following maximum deflection of the cornea and once it has folded back into the original state, the cornea passes through a second, identical applanation point. It is then possible to establish an intraocular pressure by relating a pressure of the puff of air to a temporal progression of applanation of the cornea. The measured values established using the non-contact tonometer are compared with comparative measured values established using a relatively accurately measuring applanation tonometer or contact tonometer so that an internal eye pressure approximated to the actual intraocular pressure can be derived as a result.

However, an intraocular pressure measured using a non-contact tonometer is still not accurate enough compared to a pressure measurement taken using an applanation tonometer, since a measurement is falsified by the cornea, inter alia. In order to improve measurement accuracy, it has therefore been attempted to include in the measurement by non-contact tonometer the biomechanical properties of a cornea during this measurement process and to thus establish these properties during said measurement process. To this end, a puff of air is applied to a cornea, a pump pressure being measured continuously over the course of the measurement by means of a pressure transducer. A temporal progression of the measurement is also monitored, and a first and a second applanation point of the cornea are detected optically. For example, an intraocular pressure can be derived by determining the pressures prevailing at the moment of first and second applanation, in particular since the forces required to curve the cornea when the cornea is folded inwardly and outwardly are to be assumed to be of identical magnitude and thus cancel each other out. An intraocular pressure consequently results from an average of the force, applied by the puff of air, used to fold the cornea inwardly and outwardly.

It is alternatively known to determine a hysteresis between the first and second applanation points and to derive or correct the intraocular pressure on the basis of the hysteresis measurement. A disadvantage of this measurement method is that a movement of the cornea caused by a puff of air is subject to dynamic effects, which may falsify time/pressure measurements of this type, in particular since the dynamic effects in the case of the described non-contact tonometer measurements cannot be taken into account.

Overall, the analysis methods and systems known from the prior art with parallel interdependent pressure and time measurement with simultaneous detection of the applanation points are therefore still relatively inaccurate compared to a measurement taken using a contact tonometer. Even if the aforementioned, possible error sources are taken out of consideration for inaccuracies, there are clearly still further measurement falsifying effects in the case of non-contact tonometer measurements of this type.

### SUMMARY OF THE INVENTION

The object of the present invention is therefore to propose an ophthalmological analysis method for measuring an intraocular pressure in an eye and an analysis system of this type, with which a comparatively improved measurement accuracy can be achieved.

This object is achieved by an ophthalmological analysis method including the steps of applying a puff of air to an undeformed cornea of the eye using the actuation device to deform the cornea, monitoring and recording deformation of the cornea; recording sectional images of the undeformed and deformed cornea; and deriving an intraocular pressure from the sectional images of the cornea, wherein the recorded sectional images of the deformed cornea are corrected relative to a recorded sectional image of the undeformed cornea, the intraocular pressure being derived under consideration of the correction. The object is also achieved by an analysis system having an actuation device applying a puff of air to an undeformed cornea of the eye to deform the cornea; a monitoring system monitoring and recording the deformation of the cornea, said monitoring system recording sectional images of the undeformed and deformed cornea; and an analysis device deriving an intraocular pressure from the sectional images of the cornea, wherein the recorded sectional images of the deformed cornea are corrected relative to a recorded sectional image of the undeformed cornea, the intraocular pressure being derived under consideration of the correction.

In the ophthalmological analysis method according to the invention for measuring an intraocular pressure in an eye using an analysis system, the analysis system comprises an actuation device, with which a cornea of the eye is deformed contactlessly, a puff of air being applied to the eye using the actuation device to deform the cornea, comprises a monitoring system with which the deformation of the cornea is monitored and recorded, sectional images of the undeformed and deformed cornea being recorded using the monitoring system, and comprises an analysis device with which the intraocular pressure is derived from the sectional images of the cornea, wherein the recorded sectional images of the deformed cornea are corrected relative to a recorded sectional image of the undeformed cornea, the intraocular pressure being derived under consideration of the correction.

Surprisingly, it has been found when monitoring an entire system of the eye over the course of a non-contact tonometer measurement that there are differences between a recorded sectional image of the undeformed cornea and sectional images of the deformed cornea with regard to a relative position. A force is thus applied to the cornea of the eye as a result of the application of the puff of air and causes a movement of the entire eye. Since, in the case of the non-contact tonometer measurements known from the prior art, merely a front region of the eye is monitored, a movement of the entire system of the eye cannot be taken into account when deriving the intraocular pressure, which leads to falsification of the measured values established. In accordance with the analysis method according to the invention, the recorded sectional images of the deformed cornea, which are falsified by a movement of the entire system of the eye relative to a sectional image of the undeformed cornea, are thus corrected by the errors in question and only then is the intraocular pressure derived from the recorded sectional images of the deformed and undeformed cornea. An error source, which was not previously taken into account when measuring the intraocular pressure by means of a non-contact tonometer, can thus be eliminated effectively, and a much improved level of measurement accuracy is achieved.

The sectional image of the undeformed cornea can thus be used as a reference point for the sectional images of the deformed cornea. Consequently, a spatial deviation of the sectional images of the deformed cornea, caused by a movement of the entire system of the eye, from the reference point or from at least one sectional image of the undeformed cornea can be corrected. The sectional image of the undeformed cornea or a position of the undeformed cornea to be inferred therefrom is therefore used as a reference point for the spatial deviation of the deformed cornea and of the entire system of the eye.

The sectional images of the deformed cornea can thus be corrected since the sectional images of the deformed cornea are each corrected relative to the sectional image of the undeformed cornea by a spatial offset. A movement of the entire system of the eye caused by the puff of air in a direction away from the actuation device consequently causes a spatial, parallel offset of the entire eye in the direction of an optical axis or device axis of the analysis system. The errors thus occurring during a measurement can be corrected particularly easily by establishing the offset. It is then merely necessary to correct the sectional images of the deformed cornea by the spatial offset relative to the sectional images of the undeformed cornea.

A correction of this type can be improved further still if a function of the offset is taken into account. This means that a movement of the entire eye in relation to a period of deformation of the cornea does not necessarily progress parallel and linearly to the deformation of the cornea. For example, it can be taken into account during correction that a movement of the entire eye is delayed in relation to a movement of the cornea owing to the differences between the respective masses, and a maximum of possible offset is not yet reached, even when maximum deformation of the cornea has been reached. It may also be taken into account that the movement of the entire system of the eye does not extend linearly in relation to a deformation period of the cornea, since an eye socket accommodating the eye already counters the movement of the eye with a resistance once the puff of air has been applied to the eye.

A period of time between the start and end of deformation of the cornea can also be measured. In particular, all recorded sectional images can thus be assigned to a respective specific moment of the measurement, whereby a temporal course of the deformation can be reproduced. In particular, a moment of first and second applanation of the cornea and therefore a time interval can be determined accurately. The establishment of this period of time can also be used to determine the relevant correction value. Furthermore, a period of time of the entire deformation of the cornea can be consulted in order to derive the correction value.

A speed of the moved cornea can also be measured. In particular if the temporal progression of a deformation of the cornea is known, the dynamics of the deformation or of the offset can thus also be examined so as to assess particular dynamic effects during deformation with regard to the necessary correction. For example, a post-vibration of the cornea upon application of a puff of air therefore can no longer have a falsifying effect on a measurement result if the post-vibration is taken into account in the measurement. A speed of a puff of air is thus also selectable arbitrarily in relation to dynamic effects which are otherwise undesired for a measurement. It is also possible to deduce from the measured speed a press-in depth or maximum amplitude of the deformation and of the offset, since there is a functional relationship between these variables.

A particularly accurate correction is possible if an offset of an eyeball is measured. A movement of the entire system of the eye can thus be detected in its entirety and therefore corrected.

It is also possible to measure an offset of an ocular fundus. For example, an interferometer or another suitable measuring device can be used to determine an eye length or a distance of an ocular fundus or a point of a retina of the eye in relation to the measuring device. This distance can be measured continuously during the deformation of the cornea by means of the puff of air, whereby an offset of the retina caused by the puff of air can be established. A movement of the entire system of the eye can thus be measured substantially relatively accurately.

To this end, it is also possible to derive an offset from the sectional images of the deformed and undeformed cornea, either alone or in addition to the above-described modifications to the method. If a series of sectional images of the deformed cornea are recorded, it is possible to determine, on the basis of the progression of deformation which emerges from the sectional images, whether an offset of the eye occurs as a result of the puff of air, and how large this offset is.

This offset can be established from a plurality of reference points in an edge region of the sectional images remote from an optical axis or device axis. The cornea is deformed by the puff of air to a much lesser extent in an edge region of a recorded sectional image, for example in a transition region to a sclera of the eye. Rather, a deformation emerging from a comparison of the sectional images results from an offset of the respective corneal region caused by a movement of the entire system of the eye. If any influence or effect of an offset on a deformation of the edge region of the cornea is known, this can be taken into account to correct the sectional images of the deformed cornea.

It is further advantageous if a maximum offset is established. The moment at which the deformation of the cornea achieves a maximum offset of the entire system of the eye can thus be established easily. These measured values can also be consulted for a more accurate correction of the recorded sectional images of the deformed cornea.

It should also be noted that, in the case of the method according to the invention, it is not necessary to measure the pressure of a pump pressure. Any measurement of an intraocular pressure can thus always be carried out at the same constant pump pressure. Since the level of the pump pressure and the temporal synchronisation of the pump pressure do not have to be varied in this case, a range of possible error sources can be eliminated and a particularly accurate measurement can be taken.

It is further advantageous if a pump pressure for producing the puff of air progresses in the form of a bell curve in relation to a duration thereof. The pump pressure can thus act on the cornea in the form of the puff of air, identically for each individual measurement and completely uninfluenced. The bell curve may have a symmetrical shape, inter alia.

A maximum pump pressure for producing the puff of air may also be identical in previous and subsequent measurements. A particularly good comparability of different measurements can thus be enabled. The maximum pump pressure may be 70 mm Hg for example.

In order to still be able to correct a pump pressure where necessary and to check a desired pressure curve, a pump pressure for producing the puff of air can be measured once an applanation point of the cornea is reached. For example, a pump may have a pressure sensor which makes it possible to monitor the pump pressure over the entire course of the measurement. Any errors with regard to the pump pressure can be eliminated during the measurement and continuity of successive measurements can be ensured.

In order to determine a correction value more precisely, a maximum deformation of the cornea can be derived from the sectional images of the cornea. A maximum press-in depth of the cornea can thus be established from the sectional images, wherein a moment of maximum deformation of the cornea can also be determined, at least in relation to one of the applanation points.

The necessary correction of the sectional images of the cornea can be determined more accurately if an amplitude of the deformation of the cornea is derived from the sectional images of the cornea. The precise geometrical progression of the deformation and of the offset can thus be reproduced easily. This means that, at any moment of the deformation, the geometrical form of the deformation at this moment can be recorded, and therefore the geometrical progression of the deformation can be measured in the manner of a film of the deformation. For example, a post-vibration of the cornea once it has been folded outwardly or after a second applanation point can also be measured effectively.

A size of a planar applanation area can also optionally be measured, even when an applanation point of the cornea is reached. For example, a size of the applanation area and/or the diameter thereof and/or the shape thereof may be taken into account as an indicator for a waypoint of the deformation of the cornea.

Furthermore, the deformation area or applanation area may be consulted in a specific time period of the deformation in relation to another measurable point or offset of the cornea during the deformation to define the offset of the cornea or of the sectional images as a result of a movement of the eye. The established deviation and relative values of the respective position can also be stored and compared in a database. An objective internal pressure of the eye or a corresponding correction value can thus be known for the values stored in the database, and therefore the objective intraocular pressure of the measured eye can be derived under consideration of the offset of the cornea or of the entire eye.

The offset of the sectional images of the cornea can be differentiated further still if the deformation of the cornea is continued by a free vibration of the cornea, and if a further correction of the free vibration of the cornea takes place. Consequently, sectional images of the cornea beyond the actual deformation of the cornea can be recorded by means of the monitoring system so as to establish any free vibration of the cornea.

In an advantageous embodiment of the analysis method, the monitoring system may comprise a camera and an illumination device in a Scheimpflug arrangement, wherein the sectional images can be recorded by means of the camera. This means that the camera may be arranged in a Scheimpflug arrangement relative to an optical axis of a gap illumination device for illuminating the eye, so that an illuminating cross-sectional image of the eye can be recorded using the camera. For example, a camera may also be used as a high-speed camera which can take at least 4000 images per second. The optical axis of the gap illumination device may also fall within an optical axis of the eye or coincide therewith. An active direction of the puff of air may then preferably extend coaxially with the optical axis of the gap illumination device.

The ophthalmological analysis system according to the invention for measuring an intraocular pressure in an eye comprises an actuation device, with which a cornea of the eye can be deformed contactlessly, it being possible to apply a puff of air to the eye using the actuation device to deform the cornea, comprises a monitoring system with which the deformation of the cornea can be monitored and recorded, sectional images of the undeformed and deformed cornea being recorded using the monitoring system, and comprises an analysis device with which the intraocular pressure can be derived from the sectional images of the cornea, wherein the recorded sectional images of the deformed cornea are corrected relative to a recorded sectional image of the undeformed cornea, the intraocular pressure being derived under consideration of the correction.

### BRIEF DESCRIPTION OF THE DRAWINGS

A preferred embodiment of the invention will be described in greater detail hereinafter with reference to the accompanying drawings, in which:

FIGS. 1a to 1c: show longitudinal sectional views of deformation of a cornea of an eye during a measurement process;

FIG. 2: shows a graph illustrating pump pressure and pump time during a measurement process; and

FIG. 3 shows a schematic view of an ophthalmological analysis system incorporating the present invention.

### DETAILED DESCRIPTION OF THE EXAMPLE EMBODIMENTS

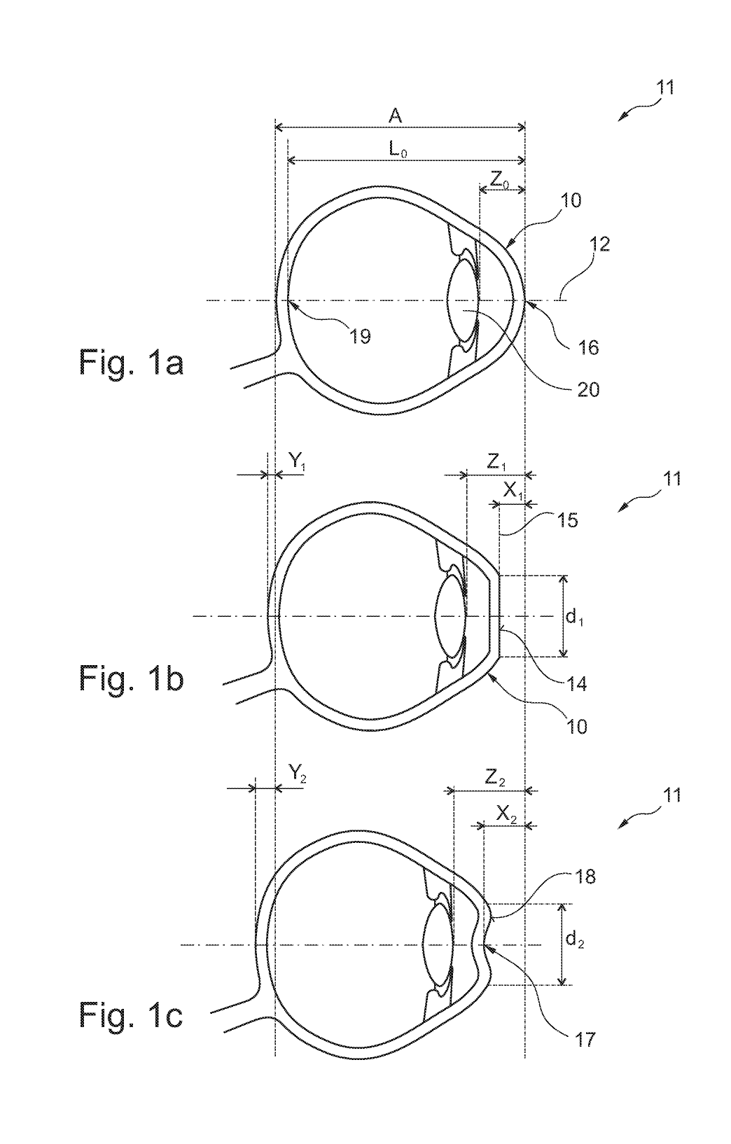

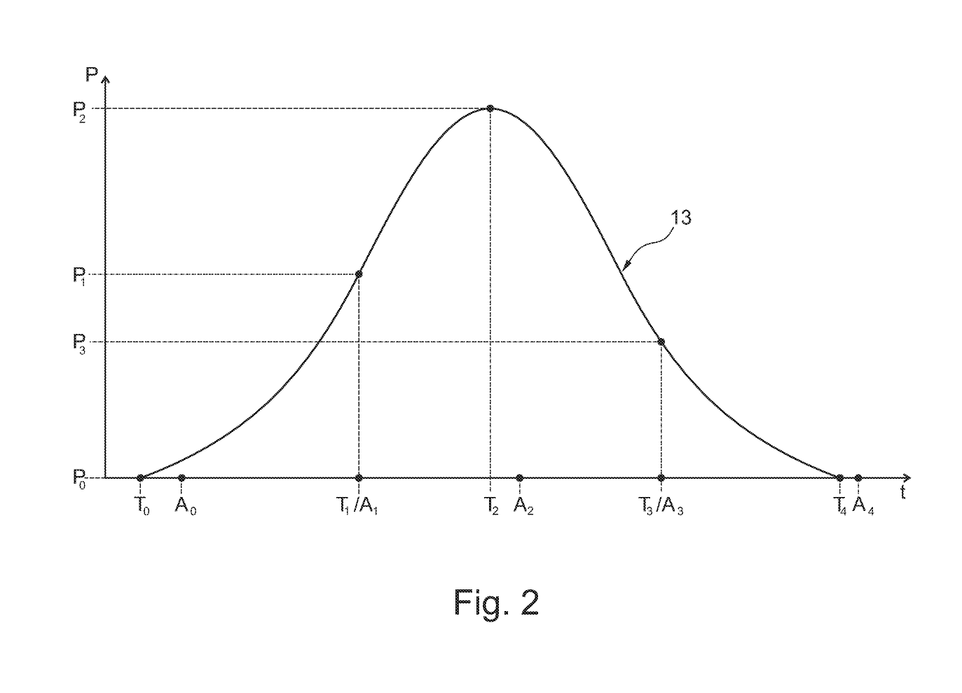

FIGS. 1a to 1c show selected states of deformation of a cornea 10 of an eye 11 during an individual measurement of an internal eye pressure using an analysis system 22 shown in FIG. 3. The illustrations of FIGS. 1a to 1c are longitudinal sectional illustrations along an optical axis 12 of the eye 11. FIG. 2 shows a graph illustrating a time t on the abscissa axis and a pump pressure p of an actuation device 26 on the ordinate axis. Independently of use of a monitoring system, such as a Scheimpflug camera 24 having a gap illumination device 28, the pump pressure progresses in the manner of a symmetrical bell curve 13, starting at a pressure P₀ at a start time T₀ of the pump up to a maximum pump pressure P₂ at a time T₂, and then falling again as far as the pump pressure P₀ at an end time T₄. The puff of air discharged by the actuation device 26 onto the cornea 10 at T₀ by starting the pump leads to a first deformation of the cornea 10, which can be recorded by the monitoring system 24, directly after the time A₀. FIG. 1a shows the shape of the cornea 10, which is not yet deformed, at the time A₀. With increasing pump pressure, complete applanation of the cornea 10 in accordance with FIG. 1b is observed at time A₁, wherein, as illustrated here, an applanation area 14 of diameter d₁ is formed which is substantially planar and lies in a plane of applanation 15. The cornea is then removed or pressed in from the apex 16 of the cornea 16 by a measure X₁. A pump pressure P₁ may optionally, and not necessarily, be established at the coinciding time T₁ at the moment said first applanation point is reached at time A₁. Once the pump pressure P₂ has been reached, there is maximum deformation of the cornea 10 at time A₂, corresponding to the illustration in FIG. 1c. A point 17 determining maximum deformation is removed from the apex 16 of the cornea 10 by a measure X₂. In this case, this is thus a maximum deflection of an amplitude of the deformation. A diameter d₂ of a concave deformation area 18 is formed and measured at this maximum amplitude of deformation. The diameter d₂ is defined by a distance between two opposed points of a plane of longitudinal section of the cornea 10, wherein the points represent the closest points of the cornea 10 facing the analysis system 22. The cornea 10 then carries out a return movement or stops vibrating, wherein the second applanation point is reached at the time A₃, this not being illustrated here in greater detail. It is also optionally possible to determine a pump pressure P₃ at the coinciding time T₃. Once the pump pressure has returned to the original value P₀ at time T₄, the cornea 10 reaches its starting position again, illustrated in FIG. 1a, at time A₄. The described states of deformation of the cornea 10, which are characterised by the respective times denoted by A₀ to A₄, are established in accordance with the above description of an individual measurement of intraocular pressure of an eye. The time intervals of the relevant times A₀ to A₄ and the measures or press-in depths X₁ and X₂ are measured in particular independently of a pump pressure p.

As can be further inferred from FIGS. 1a to 1c, the eye 11 has an eye length A and a distance from the apex 16 to a retina 19 along the optical axis 12 with a length L₀. A length Z₀ from the apex 16 to a lens 20 can also be measured. For example, the length Z₀ can be measured by means of a camera in a Scheimpflug arrangement, and a length L₀ can be measured using an interferometer. When the cornea 10 is deformed by means of the puff of air, as shown in FIG. 1b, the entire eye 11 is offset in an eye socket (not illustrated) along the optical axis 12 by the length Y₁. Since the cornea 10 is deformed measurably by the press-in depth X₁, there is actual deformation of the cornea 10 relative to the apex 16 according to the equation X_{correction}=X₁−Y₁. Consequently, the sectional image shown in FIG. 1b is corrected by the length Y₁ in order to use the corrected sectional image to derive the intraocular pressure. If the cornea 10 is deformed further up to the press-in depth X₂, the eye 11 is likewise offset further by the length Y₂. The sectional image of the deformed eye 11 shown in FIG. 1c is then shifted or corrected along the optical axis 12 by the length Y₂, as described previously. As an alternative or in addition, the respective sectional images may also be corrected similarly on the basis of the differences between the lengths Z₀, Z₁ and Z₂.

With use of sectional images of the deformed eye 11 and of the cornea 10 corrected in such a way, it is possible to eliminate a substantial error source when deriving the intraocular pressure from the sectional images of the cornea and to thus obtain a measurement of intraocular pressure which is more accurate compared to the measurement methods known from the prior art.

1. An ophthalmological analysis method for measuring an intraocular pressure in an eye, said method comprising:
recording at least one sectional image of an undeformed cornea of the eye;
applying a puff of air to the undeformed cornea of the eye to deform the cornea and offset the entire eye along an optical axis;
recording at least one sectional image of the deformed cornea;
deriving the offset of the entire eye along the optical axis from a comparison of a position of the eye in the at least one sectional image of the deformed cornea relative to a position of the eye in the at least one sectional image of the undeformed cornea;
correcting the at least one sectional image of the deformed cornea by the offset; and
deriving the intraocular pressure from the sectional images of the deformed and undeformed cornea as corrected for the offset of the entire eye along the optical axis.

2. The analysis method according to claim 1, in which the sectional image of the undeformed cornea is used as a reference point for a plurality of sectional images of the deformed cornea to derive the offset of the entire eye along the optical axis.

3. The analysis method according to claim 1, in which a function of the offset is taken into account when correcting the intraocular pressure.

4. The analysis method according to claim 1, in which an offset of an ocular fundus is measured to determine the offset of the entire eye along the optical axis.

5. The analysis method according to claim 1, in which the offset derived from the at least one sectional image of the deformed cornea relative to the undeformed cornea is established from a plurality of reference points in an edge region of the at least one sectional image of the deformed cornea relative to the at least one sectional image of the undeformed cornea remote from one of the optical axis and a device axis to determine the offset of the entire eye along the optical axis.

6. The analysis method according to claim 1, in which a maximum offset of the eye resulting from deformation of the cornea caused by applying the puff of air to the undeformed cornea of the eye is measured to determine the offset of the entire eye along the optical axis.

7. The analysis method according to claim 1, in which a pump pressure for producing the puff of air progresses in a form of a bell curve in relation to a duration thereof.

8. The analysis method according to claim 7, further comprising the step of applying a second puff of air, recording at least one sectional image of the deformed cornea after the second puff of air is applied, and deriving the offset of the entire eye along the optical axis from a comparison of a position of the eye in the at least one sectional image of the deformed cornea after application of the second puff of air and the relative to a position of the eye in the at least one sectional image of the undeformed cornea, wherein a maximum pump pressure for producing the second puff of air is identical to the maximum pump pressure for producing the initial puff of air.

9. The analysis method according to claim 7, in which the pump pressure for producing the puff of air is measured once an applanation point of the cornea is reached.

10. The analysis method according to claim 1, in which a maximum deformation of the cornea is derived from a comparison of the at least one sectional image of the deformed cornea relative to the at least one sectional image of the undeformed cornea.

11. The analysis method according to claim 1, in which the at least one sectional image of the deformed cornea is recorded using a camera in a Scheimpflug arrangement.

12. The analysis method according to claim 1, wherein the step of applying the puff of air to the undeformed cornea of the eye moves the entire eye along the optical axis inside an eye socket.
